(19) United States
(12) Patent Application Publication
(10) Pub. No.: US 2013/0338198 A1
(43) Pub. Date: Dec. 19, 2013

(54) COMPOSITION FOR TREATING DERMATITIS AND ICHTHYOSIS, AND METHOD FOR TREATING DERMATITIS AND ICHTHYOSIS

(71) Applicant: James A. Roszell, Las Vegas, NV (US)

(72) Inventor: James A. Roszell, Las Vegas, NV (US)

(73) Assignee: Skinvisible Pharmaceuticals, Inc., Las Vegas, NV (US)

(21) Appl. No.: 13/992,498

(22) PCT Filed: Dec. 9, 2011
(86) PCT No.: PCT/US11/64190

§ 371 Date: Aug. 21, 2013

Related U.S. Application Data
(60) Provisional application No. 61/421,823, filed on Dec. 10, 2010.

(51) Int. Cl. A61K 31/401

(52) U.S. Cl. CPC A61K 31/401
USPC 514/350

(57) ABSTRACT

A skin disorder treatment composition and method for treating a skin disorder or symptoms of a skin disorder are provided. The composition includes: about 0.0001 wt. % to about 5 wt. % of a serine protease inhibitor; about 2 wt. % to about 10 wt. % of a hydrophobic polymer/hydrophilic polymer complex comprising a poly(vinylpyrrolidone-alkylene) polymer wherein the alkylene group contains at least 10 carbon atoms and a cellulose containing polymer; about 0.5 wt. % to about 20 wt. % of a release agent; and at least about 50 wt. % water. The skin disorder or the symptom of skin disorder from ichthyosis, dermatitis, eczema, rosacea, psoriasis, inflammation, xerosis, or urticaria.

This application is being filed on 9 Dec. 2011, as a PCT International Patent application in the name of Skinvisible Pharmaceuticals, Inc., a U.S. national corporation, applicant for the designation of all countries except the U.S., and, James A. Roszell, a citizen of the United States, applicant for the designation of the U.S. only, and claims priority to U.S. Patent Application Ser. No. 61/421,823 filed on Dec. 10, 2010, the disclosure of which is incorporated herein by reference in its entirety.

### FIELD OF THE INVENTION

The invention relates to compositions for treating skin disorders and to methods for treating skin disorders. Exemplary skin disorders include dermatitis, ichthyosis, eczema, rosacea, psoriasis, inflammation, xerosis, urticaria, or symptoms thereof. The dermatitis can be a form of “diaper rash” caused by the presence of proteolytic enzymes in body waste such as urine or feces. The ichthyosis can be caused by a genetic disorder resulting in a lack of serine protease inhibitor in skin and can be a form of Netherton Syndrome.

### BACKGROUND

Incontinence is common in infants, senior citizens, and colostomy patients. In the cases of infants and seniors, the problem is frequently handled by using diapers or absorbent undergarments. Rather than eliminating dermatitis, diapers and absorbent undergarments tend to contribute to dermatitis by holding urine and feces in close contact with skin until they are removed, and the skin is cleaned.

Much of the dermatitis or diaper rash is caused by the presence of proteolytic enzymes. Some of the proteolytic enzymes are generated by the person's intestinal tract as a result of digesting food. Other proteolytic enzymes are secreted by bacteria to digest an exogenous food source. The bacteria consider skin to be a food source, and the bacteria produce an abundant supply of proteolytic enzymes to digest this food source.

Many of the digestive enzymes in a person's intestines and produced by bacteria are Serine proteases. The most familiar human serine proteases are trypsin, chymotrypsin, and elastase. A common bacterial serine protease is subtilisin. Not all proteases are for digestive purposes. There are many proteases in a person's body that are involved in immune systems, blood clotting, and skin exfoliation. As nature frequently does when producing something as potentially destructive as proteases, nature also produces protease inhibitors. In the cases of the immune system, blood clotting, and skin exfoliation, the inhibitors and their respective proteases exist in balance. When this balance is disturbed, undesirable changes can result.

For example, one of the basic structural components of our skin is elastin. Elastin is responsible for maintaining the smooth, youthful elastic properties of skin. With age, a person produces more elastase, the serine protease that digests elastin, and less elastin inhibitor, resulting in a decrease in the elastic properties of the skin, and the development of fine lines and wrinkles. Since elastase is a serine protease, supplying exogenous serine protease inhibitors should slow the degenerative process of skin aging.

One of the best known serine protease inhibitors is soy bean trypsin inhibitor (SBT). SBT is a non-specific protease and would be expected to inhibit any serine protease. SBT is a large glycoprotein, produced in soy beans, that inhibits the digestive action of trypsin. Most people do not eat enough soy bean products for SBT to interfere with digestion, however, it is possible that the presence of SBT in soy bean products is the reason soy is so highly touted as a diet aid. Theoretically, by inhibiting the digestion of proteins in the gut, their absorption into the body can be decreased. SBT is likely not useful for reducing skin aging by inhibiting elastase because SBT has a molecular weight that is too high to penetrate the skin. It is generally assumed that materials with a molecular weight greater than 1000 do not penetrate the skin. There are several synthetic serine protease inhibitors, but they are generally too toxic to apply to skin.

A type of ichthyosis called Nethertone Syndrome is believed due to a genetic defect in the production of a specific serine protease inhibitor. Patients suffering from Nethertone Syndrome often exhibit symptoms that include cracking/scaling of skin, fragile and spiky hair, and allergies to nuts and fish.

### SUMMARY

A dermatitis treatment composition is provided according to the present invention. The dermatitis treatment composition includes: about 0.0001 wt. % to about 5 wt. % of a serine protease inhibitor; about 2 wt. % to about 10 wt. % of a hydrophobic polymer/hydrophilic polymer complex comprising a poly(vinylpyrrolidone-alkylene) polymer wherein the alkylene group contains at least 10 carbon atoms and a cellulose containing polymer; about 0.5 wt. % to about 20 wt. % of a barrier component; and at least about 50 wt. % water.

A method for treating dermatitis is provided according to the present invention. The method includes a step of applying a dermatitis treatment composition to skin tissue.

An ichthyosis treatment composition is provided according to the present invention. The ichthyosis treatment composition includes: about 0.0001 wt. % to about 5 wt. % of a serine protease inhibitor; about 2 wt. % to about 10 wt. % of a hydrophobic polymer/hydrophilic polymer complex comprising a poly(vinylpyrrolidone-alkylene) polymer wherein the alkylene group contains at least 10 carbon atoms and a poly(maleic acid/methylvinylether) copolymer; about 0.5 wt. % to about 5 wt. % of a release agent; and at least about 50 wt. % water.

A method for treating ichthyosis is provided according to the present invention. The method includes a step of applying an ichthyosis treatment composition to skin tissue.

A composition for treating skin disorders or symptoms of skin disorders is provided according to the present invention. The skin disorders can include symptoms of dermatitis, ichthyosis, eczema, rosacea, psoriasis, inflammation, xerosis, or urticaria. The composition includes: about 0.0001 wt. % to about 5 wt. % of a serine protease inhibitor; about 2 wt. % to about 10 wt. % of a hydrophobic polymer/hydrophilic polymer complex comprising a poly(vinylpyrrolidone-alkylene) polymer wherein the alkylene group contains at least 10 carbon atoms and a cellulose containing polymer; about 0.5 wt. % to about 20 wt. % of a release agent; and at least about 50 wt. % water.

A method for treating skin disorders or symptoms of skin disorders is provided according to the present invention. The method includes steps of applying the treatment composition to skin tissue.

### BRIEF DESCRIPTION OF THE DRAWINGS

FIG. 1 is a graph showing recovery of trypsin activity after exposure to DPHP according to example 1.

FIG. 2 is a graph showing the Lineweaver-Burk plot of uninhibited trypsin using N-benzoyl-L-arginine ethylester as a substrate and the inhibition of trypsin by DPHP.

### DETAILED DESCRIPTION

A topical composition is provided that can be used for treating dermatitis, or symptoms of dermatitis, including dermatitis resulting from contact by skin tissue by body waste such as urine and feces. Such dermatitis can be referred to as diaper rash because it is commonly found in infants and seniors who use diapers or absorbent undergarments. The topical composition for treating dermatitis can be referred to as a dermatitis treatment composition. The dermatitis treatment composition can provide barrier properties. A composition provides urine and feces barrier properties when the composition, after being applied to skin, prevents urine and feces from passing through it and contacting the skin. In general, the dermatitis treatment composition can provide barrier properties that are at least as good as the barrier properties of commercially available zinc oxide ointment.

The dermatitis treatment composition includes a serine protease inhibitor, a skin bonding polymer component, and a barrier component. The skin bonding polymer component holds the serine protease inhibitor together with the barrier component, and the barrier component is at least in part responsible for providing barrier properties when the dermatitis treatment composition is applied to skin. For example, the dermatitis composition treatment composition can be applied to skin where the dermatitis or diaper rash is likely to occur. By providing barrier properties, the dermatitis treatment composition reduces the likelihood of proteases contacting the skin and causing dermatitis or diaper rash. In addition, the presence of the of serine protease inhibitor within the composition helps reduce the harmful affects of the protease in the urine and feces.

A topical composition is provided that can be used for treating ichthyosis such as Nethertone Syndrome type ichthyosis or symptoms of ichthyosis. It is believed that Nethertone Syndrome type ichthyosis can be due to a genetic defect in the production of a specific serine protease inhibitor. As a result of the lack of a specific serine protease inhibitor, patients suffering from Netherone Syndrome can have chronic allergy problems due to the lack of integrity of their skin. A composition that can be used for treating ichthyosis can be referred to as an ichthyosis treatment composition. In general, the ichthyosis composition includes a serine protease inhibitor, a skin bonding polymer component, and a release agent. The release agent helps the serine protease inhibitor move from the skin bonding polymer component to skin over time.

Methods for treating dermatitis and ichthyosis include applying a dermatitis treatment composition or an ichthyosis treatment composition to skin tissue. In the case of treating dermatitis, the dermatitis treatment composition can provide barrier properties to prevent urine and feces from passing therethrough and contacting the skin tissue. The presence of a serine protease inhibitor in the dermatitis treatment compositions helps reduce the harmful affect that proteases present in urine and feces may have on the skin tissue if it does, over time, pass through the composition. The method for treating ichthyosis can include applying the ichthyosis treatment composition to skin tissue that is affected by ichthyosis or exhibits symptoms of ichthyosis.

A topical composition is provided that can be used for treating dermatitis, ichthyosis, eczema, rosacea, psoriasis, inflammation, xerosis, and urticaria, or symptoms of dermatitis, ichthyosis, eczema, rosacea, psoriasis, inflammation, xerosis, and urticaria. The treatment composition includes a serine protease inhibitor, a skin bonding polymer component, and a release agent. The release agent helps the serine protease inhibitor move from the skin bonding polymer component to skin over time. A method for treating skin disorders is additionally provided. The skin disorders can include dermatitis, eczema, rosacea, psoriasis, inflammation, xerosis, and urticaria, or symptoms of dermatitis, eczema, rosacea, psoriasis, inflammation, xerosis, and urticaria. It should be understood that a missing protease inhibitor may trigger a cascade that results in symptoms of dermatitis, ichthyosis, eczema, rosacea, psoriasis, inflammation, xerosis, and urticaria. The release component allows the protease inhibitor to release from the composition and penetrate into the skin tissue.

Serine Protease Inhibitor

The serine protease inhibitor that can be used includes those serine protease inhibitors that are sufficiently nontoxic when used in either the dermatitis treatment composition or the ichthyosis treatment composition. An example of a nontoxic serine protease inhibitor is dipalmitoylhydroxyproline (DPHP) sold by Seppic as Sepilift™. DPHP is recognized as having the following structure:

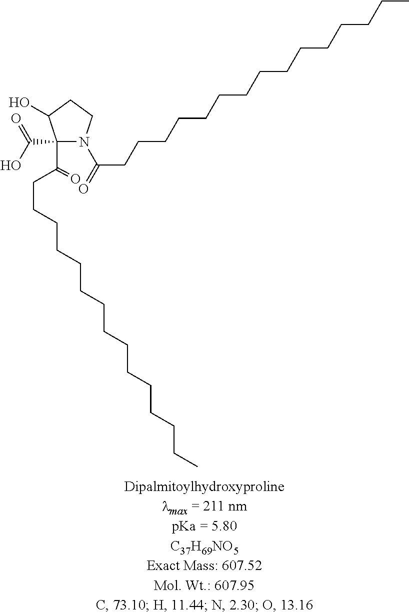 [SMILES: CCCCCCCCCCCCCCCC(=O)N1CCC(O)[C@]1(C(=O)O)C(=O)CCCCCCCCCCCCCCC]

DPHP is a derivative of a naturally occurring amino acid, hydroxyproline. This amino acid occurs primarily in connective tissue, collagen, and elastin among others. Two palmitic acid groups have been attached to the hydroxyproline to produce the DPHP. It has been shown to inhibit elastase activity. As a result, DPHP is marketed to the cosmetic market as an anti-aging ingredient.

DPHP is a non-specific serine protease inhibitor that inhibits trypsin and other serine proteases. As a result, DPHP can be provided as an active component of the topical composition for treating dermatitis associated with incontinence. To prevent contamination of care givers, the topical composition can be provided as a spray. DPHP can also be used as an exemplary serine protease inhibitor for the ichthyosis treatment composition.

The amount of serine protease inhibitor provided in the dermatitis treatment composition and the ichthyosis treatment composition can be provided in an amount of at least 0.0001 wt. % and preferably about 0.2 wt. % to about 5 wt. %, about 0.5 wt. % to about 3 wt. %, or about 0.8 wt. % to about 2.5 wt. %. When the serine protease inhibitor is DPHP, the amount of DPHP provided in either the dermatitis treatment composition or the ichthyosis treatment composition can be provided in the identified ranges.

Polymer Component

The polymer component of the topical composition can be provided as a component that exhibits a tendency to bond to skin tissue, and hold the serine protease inhibitor in place and allow the serine inhibitor to be released at a desired rate. When the polymer component is provided as a polymer that has a tendency to bond to skin tissues, the polymer component can be characterized as a skin bonding polymer component.

The polymer component can include a hydrophobic polymer/hydrophilic polymer adduct and can include other components. Polymer components that can be used according to the invention can include the polymer components disclosed in U.S. Pat. No. 6,756,059. The entire disclosure of U.S. Pat. No. 6,756,059 is incorporated herein by reference.

The polymer component of the composition can be, at least in part, responsible for holding or isolating the serine protease inhibitor. By binding to skin tissue and holding on to the serine protease inhibitor, the polymer component can help deliver the serine protease inhibitor to the skin tissue to provide a desired level of activity for a desired length of time. For example, the dermatitis treatment composition can be provided so that it adheres or binds to skin tissue for at least about one hour, and preferably at least about two hours, and holds the serine protease inhibitor for that length of time so that the serine protease inhibitor remains active for that length of time.

The polymer component can be prepared from a topical composition precursor. The topical composition precursor can be prepared by melt processing a hydrophobic polymer composition and a hydrophilic polymer composition to provide an interaction between the hydrophobic polymer composition and the hydrophilic polymer composition. It should be understood that the phrase “melt processing” refers to mixing the hydrophobic polymer composition and the hydrophilic polymer composition under conditions that provide that the hydrophobic polymer component of the hydrophobic polymer composition and the hydrophilic polymer component of the hydrophilic polymer composition are in a fluid (i.e., liquid) state so that they sufficiently mix. When the polymers are sufficiently mixed, it is believed that an interaction forms between the hydrophobic polymer component and the hydrophilic polymer component. The melt processing temperature can be at least about 50° C. and can be at least about 70° C. to generate this interaction. In addition, the melt processing temperature can be at least about 80° C. or at least above 90° C., but should not be so high that it causes degradation of the polymers.

It is theorized that the interaction exhibited between the hydrophobic polymer component and the hydrophilic polymer component is a type of complex formation reaction, and that the complex, once formed, can be stable in water at temperatures up to 65° C. and at a pH range of 3.0 to 9.0. By stable, it is meant that the complex does not favor disassociation under these conditions, and tends to exhibit a desired shelf life. It is believed that this interaction provides the composition with an ability to become emulsified in water, exhibit a desired shelf life, and bind to skin tissue. The result of the interaction between the hydrophobic polymer component and the hydrophilic polymer component can be referred to as a hydrophobic polymer/hydrophilic polymer adduct. The term “adduct” is used to refer to the interaction between the hydrophobic polymer component and the hydrophilic polymer component. The interaction may be a form of complexing, but that is only theory. Accordingly, the term “adduct” is not meant to limit the polymer component to a particular theory of interaction. The interaction, however, provides an emulsion containing the adduct with an enhanced shelf life compared with an emulsion where an adduct has not been formed. An emulsion containing the adduct can exhibit enhanced resistance to splitting into separate phases. It has been found that an emulsion formed by simply mixing the hydrophobic polymer composition and the hydrophilic polymer composition under conditions so that the adduct is not formed results in a composition that has a greater tendency to split into separate phases.

It is believed that the interaction between the hydrophobic polymer component and the hydrophilic polymer component can be achieved more easily in the absence of water. It has been found that if the hydrophilic polymer component becomes dissolved in water before forming the complex, it can be more difficult to sufficiently mix the hydrophobic polymer component and the hydrophilic polymer component to provide the desired level of interaction. Although a convenient technique for providing the desired level of interaction between the hydrophobic polymer component and the hydrophilic polymer component is melt mixing, it is expected that other techniques can be used to achieve the desired level of interaction.

The hydrophobic polymer composition includes at least one hydrophobic polymer and can include a mixture of hydrophobic polymers. The hydrophobic polymer composition can include components having repeating pyrrolidone/alkylene groups. Exemplary polymers having repeating pyrrolidone/alkylene groups include poly(vinylpyrrolidone/alkylene) polymers. Poly(vinylpyrrolidone/alkylene) polymers include those polymers obtained by polymerizing alkylene substituted vinylpyrrolidone. Poly(vinylpyrrolidone/alkylene) polymers can be represented by the following general formula:

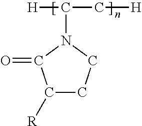

wherein R represents a carbon chain such as an alkylene group and n represents the number of repeating units. The R group is preferably sufficiently long so that the polymer remains relatively water insoluble and should not be too long so that the polymer is difficult to melt process. The alkylene group can contain at least about 10 carbon atoms and can contain less than about 30 carbon atoms. The alkylene group can contain about 14 carbon atoms to about 22 carbon atoms, and can contain about 15 carbon atoms to about 19 carbon atoms.

The poly(vinylpyrrolidone/alkylene) polymers can have a molecular weight that is sufficiently high so that the polymer maintains its water insolubility but the molecular weight should not be so high that it becomes difficult to melt process the polymer. The weight average molecular weight of the poly(vinylpyrrolidone/alkylene) polymer can be between about 3,000 and about 400,000. Another way to characterize the size of the poly(vinylpyrrolidone/alkylene) polymer is by the number of repeating units (n). In the case of a poly(vinylpyrrolidone/alkylene) polymer having a weight average molecular weight of about 6,000 to about 30,000, the poly(vinylpyrrolidone/alkylene) polymer can have about 20 to about 80 repeating units, and can have about 30 to about 50 repeating units. It should be understood that repeating units refer to the residues of vinylpyrrolidone/alkylene groups.

Exemplary poly(vinylpyrrolidone/alkylene) polymers include poly(vinylpyrrolidone/1-eicosene) and poly(vinylpyrrolidone/hexadecene). Poly(vinylpyrrolidone/1-eicosene) can be referred to as PVPE and is commonly used in pharmaceutical and cosmetic preparations. An exemplary form of PVPE for use according to the invention includes about 43 to 44 repeating units in length and has a weight average molecular weight of about 17,000 and can be characterized as a paraffin-like solid. This particular PVPE is highly insoluble in water, and has an extremely low oral toxicity (LD₅₀>17000 mg/kg) and exhibits no demonstrable dermal toxicity. Poly(vinylpyrrolidone/1-hexadecene) can be referred to as PVPH. An exemplary form of PVPH is available as a viscous yellow liquid that is insoluble in water and has a low oral toxicity (LD₅₀>64000 mg/kg), has about 39 to 40 repeating units, a molecular weight of about 14,000, and exhibits no demonstrable dermal toxicity.

PVPE and PVPH differ in the length of the hydrocarbon side chain, and are used extensively in the skin care industry, usually at concentrations of less than 1% by weight, because of their ability to bind to skin. Because the skin care industry generally prefers to apply actives to skin using a water-based composition, the use of PVPE and PVPH often requires solvents, surfactants, and emulsifiers to stabilize these polymers in a water emulsion. However, many of the solvents, surfactants and emulsifiers used to stabilize PVPE and PVPH in a water emulsion lack the low dermal toxicities of PVPE and PVPH. PVPE and PVPH by themselves lack a cosmetically elegant appeal when applied directly to the skin. They tend to be sticky and greasy.

The hydrophobic polymer composition can be provided as a single poly(vinylpyrrolidone/alkylene) polymer or as a mixture of different poly(vinylpyrrolidone/alkylene) polymers. The mixture of different poly(vinylpyrrolidone/alkylene) polymers can include at least 5 wt. % of a first poly(vinylpyrrolidone/alkylene) polymer based on the weight of the hydrophobic polymer composition. The hydrophobic polymer composition can include about 5 wt. % to about 54 wt. % of the first poly(vinylpyrrolidone/alkylene) polymer. The second poly(vinylpyrrolidone/alkylene) polymer can be provided in an amount of at least about 46 wt. % and can be in a range of about 46 wt. % to 95 wt. % based on the weight of the hydrophobic polymer composition. For a hydrophobic polymer composition containing a first poly(vinylpyrrolidone/alkylene) polymer and a second poly(vinylpyrrolidone/alkylene) polymer, the mole ratio of the first polymer to the second polymer can be about 1:22 to about 1:1. When the hydrophobic polymer composition contains a mixture of different poly(vinylpyrrolidone/alkylene) polymers, the poly(vinylpyrrolidone/alkylene) polymers can be selected to provide improved properties compared to a composition having a hydrophobic polymer composition containing a single poly(vinylpyrrolidone/alkylene) polymer.

When the hydrophobic polymer composition is provided as a mixture of PVPH and PVPE, the PVPH can be provided in a range of about 46 wt. % to about 95 wt. % and the PVPE can be provided in a range of about 5 wt. % to about 65 wt. %, based upon the weight of the hydrophobic polymer composition.

The hydrophilic polymer composition can include a poly(maleic acid/methylvinylether) copolymer or a mixture of poly(maleic acid/methylvinylether) copolymers. Poly(maleic acid/methylvinylether) copolymers that can be used can have a weight average molecular weight of at least about 50,000, and can have a weight average molecular weight of about 50,000 to about 4,000,000. The weight average molecular weight can be about 70,000 to 2,500,000. A general structural representation of a poly(maleic acid/methylvinylether) copolymer is shown below:

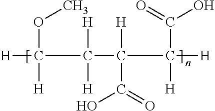

wherein n is the number of repeating units. The number n can be about 200 to about 20,000.

The hydrophilic polymer composition can include a cellulose containing polymer, and can include a mixture of cellulose containing polymers. Cellulose containing polymers that can be used include cellulose, carboxymethyl cellulose, or mixtures thereof. The hydrophilic polymer should have a molecular weight that is not too high so that the hydrophilic polymer becomes difficult to process. The weight average molecular weight of the hydrophilic polymers is preferably sufficient to provide solubility in water but not too high to become difficult to process. Cellulose that can be used can have a weight average molecular weight of about 50,000 to about 15,000,000. An exemplary cellulose component that can be used includes cellulose gum.

The melting temperature refers to the temperature at which the polymer melts, and the maximum temperature refers to the temperature at which the polymer begins to decompose. Exemplary carboxymethyl cellulose polymers that can be used include those having a melting temperature range of about 55° C. to about 60° C. and a maximum temperature range of about 75° C. to about 80° C.

The hydrophobic polymer composition and the hydrophilic polymer composition can be combined and heated to provide a melt and mixed. They can be heated to at least about 50° C. under mixing to form a complex between the hydrophobic and hydrophilic polymers. Preferably, the composition can be heated to at least about 70° C. under mixing to form a complex between the hydrophobic and hydrophilic polymers. It should be understood that a polymer melt refers to a polymer that flows or becomes fluid or liquid when heated and is not meant to refer to a polymer that forms a liquid as a result of being dissolved in a solvent.

The complex formation step can be carried out in a relatively anhydrous environment. Once the desired level of complex formation has occurred, the composition can be hydrated with water. It has been found that the presence of water during the complex formation step can have a deleterious effect on the complex formation process. The reason for this may be that water is ionic and, as a result, can have a tendency to reduce the interaction between the hydrophobic polymer component and the hydrophilic polymer component when they are melt mixed. Accordingly, it is desirable to limit the amount of water present during the complex formation step to a level that does not significantly interfere with the complex formation step or prevent the formation of a complex. In general, this level is typically less that about 1 wt. % water. Preferably, the amount of water is limited to less that about 0.5 wt. %. It should be understood that the reference to the amount of water refers to free water.

The hydrophobic polymer composition and the hydrophilic polymer composition can be mixed together in amounts sufficient to provide a ratio of pyrrolidone groups to hydrophilic groups of about 1:1 to about 5:1. The ratio of the structures causing the observed interaction between the hydrophobic polymer composition and the hydrophilic polymer composition can be referred to as “functional group parity.” The ratio of pyrrolidone groups to the combination of carboxylic acid groups and hydroxyl groups can be about 1.5:1 to about 3:1. In order to drive the complex formation reaction, it is desirable to provide an imbalance between the two types of groups. Accordingly, it is generally desirable to provide more of the pyrrolidone groups than the hydrophilic groups.

During the complex formation step, the amounts of hydrophobic polymer composition and hydrophilic polymer composition can be characterized on a weight percent basis. For example, about 2 wt. % to about 28 wt. % hydrophilic polymer composition and about 72 wt. % to about 98 wt. % hydrophobic polymer composition can be combined to provide for complex formation. About 8 wt. % to about 25 wt. % hydrophilic polymer composition and about 72 wt. % to about 95 wt. % hydrophobic polymer composition can be combined to form the complex. During the complex formation step, the amount of water available in the composition can be less than about 1 wt. %. Although the complex forming composition can be relatively anhydrous, it is expected that the amount of water can be between about 0.3 wt. % and about 1.0 wt. %. It should be understood that the amount of water refers to free water.

Once the hydrophobic polymers and the hydrophilic polymers have sufficiently reacted or interacted to form a complex, water can be added to the composition to provide a stable aqueous composition that can be relatively easily further hydrated. It has been found that the first hydration of the topical composition precursor is the most difficult hydration step because of the need to control the conditions of hydration. After the first hydration to a water content of at least about 30 wt. %, it is expected that further hydrations to higher water contents are relatively easy and can be accomplished by simply mixing the composition with water. Accordingly, the amount of water provided in the composition when made available as a concentrate for shipment is preferably between about 30 wt. % and about 45 wt. %. When the composition includes about 30 wt. % to about 45 wt. % water, it is expected that the composition can include about 3 wt. % to about 10 wt. % hydrophilic polymer composition and about 30 wt. % to about 50 wt. % hydrophobic polymer composition.

Water can be added to the relatively anhydrous composition by mixing water and the relatively anhydrous composition at a temperature and for a time sufficient to allow the composition to become hydrated without losing significant amounts of interaction between the hydrophobic polymer composition and the hydrophilic polymer composition. The relatively anhydrous composition can be hydrated by heating to at least 60° C. and adding water while mixing. The composition can be heated to at least about 65° C. and to at least about 70° C. An exemplary temperature range is about 65° C. to about 80° C.

The relatively anhydrous composition can be referred to as the topical composition precursor and generally refers to the hydrophobic polymer/hydrophilic polymer adduct containing less than about 1.0 wt. % water if any water is present. The polymer component for the composition can refer to a composition that contains only the hydrophobic polymer/hydrophilic polymer adduct, and it can refer to a composition wherein the hydrophobic polymer/hydrophilic polymer adduct is diluted with water. In general, it is desirable to have a sufficient amount of water in the polymer component that allows one to formulate the polymer component into the cationic pharmaceutically active ingredient containing composition according to the invention. If there is too little water in the polymer component, it may become difficult to formulate the composition. For example, the polymer component can contain water in an amount of up to about 95 wt. % and may contain water in an amount up to about 97 wt. %. The polymer component can have a water concentration of about 30 wt. % to about 45 wt. %.

Additional components can be added to the hydrophobic polymer/hydrophilic polymer adduct. For example, it may be desirable to add a component that helps stabilize the hydrophobic polymer/hydrophilic polymer adduct, and to help preserve and/or maintain the composition.

An exemplary polymer component that can be used is available under the name Invisicare™ C-5 composition from Skinvisible Pharmaceuticals, Inc. Invisicare™ C-5 composition contains a mixture of poly(vinylpyrrolidone/eicosene) and poly(vinylpyrrolidone/hexadecene), and carboxymethyl cellulose. Another exemplary polymer component that can be used is available under the name Invisicare™ M-1 composition from Skinvisible Pharmaceuticals, Inc. Invisicare™ M-1 composition contains a mixture of poly(vinylpyrrolidone/eicosene) and poly(vinylpyrrolidone/hexadecene), and poly(maleic acid/methylvinylether) copolymer. In the case of an incontinence treatment composition, the polymer component containing cellulose is preferable because it is more compatible with the skin of a baby or infant. In the case of an ichthyosis treatment composition, the polymer component containing poly(maleic acid/methylvinylether) copolymer is preferable because it helps assist with the release of the serine protease inhibitor. In general, when the ichthyosis treatment composition is provided at a neutral pH, the polymer component containing poly(maleic acid/methylvinylether) copolymer tends to exhibit a negative charge. The negative charge on the polymer component helps facilitate release of a negatively charged active agent.

The polymer component can be provided in the incontinence treatment composition and in the ichthyosis treatment composition in an amount of about 2 wt. % to about 10 wt. % based on the weight of the composition. In addition, the polymer component can be provided in the treatment composition in an amount of about 3 wt. % to about 8 wt. %, 4 wt. % to about 7 wt. %, and about 5 wt. % to about 6 wt. %.

Barrier Component

The dermatitis treatment composition contains a barrier component to provide the composition with barrier properties. The presence of a barrier component is desirable for treating the dermatitis or incontinence. For example, the barrier component can help reduce the passage of body waste such as urine and feces therethrough to help reduce contact of the urine and feces with skin tissue.

An exemplary barrier component is a silicone barrier component such as Dow Corning 9045. When a silicone barrier component is provided in the dermatitis treatment composition, it can be provided in an amount of about 0.5 wt. % to about 20 wt. % based on the weight of the composition. Preferably, the dermatitis treatment composition includes about 0.6 wt. % to about 5 wt. % silicone barrier component based on the weight of the composition. Other exemplary barrier components include allantoin, aluminum hydroxide gel, calamine, cocoa butter, dimethicone, glycerin, kaolin, petrolatum, shark liver oil, white petrolatum, zinc acetate, zinc carbonate, and zinc oxide. When allantoin is used as the barrier component, it can be provided in an amount of about 0.5 wt. % to about 2 wt. % based on the weight of the composition. When aluminum hydroxide gel is provided as the barrier component, it can be provided in an amount of about 0.15 wt. % to about 5 wt. %. When calamine is provided as the barrier component, it can be provided in an amount of about 1 wt. % to about 25 wt. %. When cocoa butter is the barrier component, it can be provided in an amount of 50 wt. % to about 90 wt. %. When dimethicone is provided as a barrier component, it can be provided in an amount of about 1 wt. % to about 30 wt. %. When glycerin is provided as a barrier component, it can be provided in an amount of 20 wt. % to about 45 wt. %. When kaolin is provided as a barrier component, it can be provided in an amount of about 4 wt. % to about 20 wt. %. When petrolatum is provided as a barrier component, it can be provided in amount of about 30 wt. % to about 90 wt. %. When shark liver oil is provided as a barrier component, it can be provided in amount of about 2 wt. % to about 5 wt. %. When white petrolatum is provided as a barrier component, it can be provided in an amount of 30 wt. % to about 90 wt. %. When zinc oxide is provided as a barrier component, it can be provided in amount of about 0.1 wt. % to about 2 wt. %. When zinc carbonate is provided as a barrier component, it can be provided in an amount of about 0.2 wt. % to about 2 wt. %. When zinc oxide is provided as a barrier component it can be provided in an amount of about 1 wt. % to about 25 wt. %. It should be appreciated that mixtures of various barrier components can be provided.

The ichthyosis treatment composition preferably does not include a barrier component. In general, it is desirable for the serine protease inhibitor to release from the ichthyosis treatment composition. To the extent that a barrier component would inhibit release of serine protease inhibitor from an ichthyosis treatment composition, it would be desirable to not include that barrier component in the ichthyosis treatment composition. In contrast, it is desirable for the ichthyosis treatment composition to provide a sustained release of the serine protease inhibitor. To the extent that a barrier component assists with providing a sustained release, over time, of a serine protease inhibitor from the ichthyosis treatment composition, then the ichthyosis treatment composition can include the barrier component. It should be appreciated that the listed barrier components can be specifically excluded from the ichthyosis treatment composition.

Water

The dermatitis treatment composition and the ichthyosis treatment composition can include water in an amount sufficient to allow the composition to be applied to skin tissue while providing the desired coverage over the skin tissue. The water component can be provided as deionized water, filtered water, distilled water, reverse osmosis water, or tap water. In the event that the water includes hardness or other components, it may be desirable to include builders, sequestrants, and chelating agents to handle the water hardness. In general, the composition can include at least about 50 wt. % water. In addition, it is expected that if there is too much water, the emulsion might become unstable. In general, the amount of water in the composition can be less than about 95 wt. %. The amount of water in the composition can be about 65 wt. % to about 93 wt. %.

pH Adjusting Agent

The composition can include pH adjusting agents, buffering agents, or neutralizing agents to provide the composition with a pH that helps stabilize the serine protease inhibitor. Exemplary pH adjusting agents that can be used include sodium hydroxide, potassium hydroxide, triethanolamine, acetic acid, propionic acid, citric acid, succinic acid, and mixtures thereof.

The polymer component of the lotion, cream, gel, or liquid may be at least in part responsible for reducing the irritability of the composition. For example, it is believed that the polymer component may help reduce irritation of skin tissue. The composition can be provided without any pH modifier, if desired. In general, however, a buffering agent is incorporated into the composition to help control the pH of the composition. Furthermore, the buffering agent is typically selected as a buffering agent that is compatible with skin issue or that does not harm skin tissue.

Thickener

Thickeners that can be incorporated into the composition include those components that thicken or increase the viscosity of the composition so that the composition can be readily applied to skin. Thickeners that can be used in the composition include those components often referred to as viscosity controlling agents.

Exemplary thickeners or viscosity controlling agents that can be provided in the hand disinfecting composition include cellulose gum, alkane triols; acrylates; substituted celluloses such as hydroxy ethyl cellulose, carboxymethyl cellulose, methylcellulose, and hydroxypropyl cellulose; cetyl alcohol; gums such as natural gums or synthetic gums; long chain alcohols such as those having about 9 to about 24 carbon atoms; polyglycols such as polyethylene glycols, polypropylene glycols, polybutylene glycols, polyethylene propylene glycols, or mixtures thereof; waxes such as natural waxes or synthetic waxes; hydrogenated oils; glycol esters; fatty acid esters; long chain acids; acid amides; silicates; and mixtures thereof. Exemplary thickeners that can be used is hydroxyethyl cellulose. An exemplary thickener that can be used is a polyacrylic acid thickeners available under the name Carbopol, such as, Carbopol Ultrez-10, from Lipscomb.

The composition may or may not include a thickener. When the composition includes a thickener, the thickener can be provided in an amount that provides the desired level of thickening. The composition can include a thickener in an amount of least about 0.1 wt. % and can include a thickener in an amount of at least about 0.4 wt. %. In addition, the thickener can be provided in an amount of less than about 4 wt. %, and can be provided in an amount of less than about 2 wt. %.

Release Agent

The ichthyosis treatment composition can include a release agent to assist with the sustained release of the serine protease inhibitor over a prolonged period of time. In general, a release agent is not needed for the dermatitis treatment composition and can be excluded therefrom is desired. The dermatitis treatment composition preferably exhibits barrier properties and the inhibitor is preferably contained in the composition in the event that urine or feces contacts the composition.

The release agent can be provided as a surfactant. A surfactant can additionally be present to help maintain the composition as an emulsion. In general, an emulsion refers to a composition that resists phase separation after sitting at room temperature for a couple of months. In general, it is desirable for the composition to be stored in a warehouse or in a storage closet for at least two months and can remain as an emulsion during that two month period. Preferably, the composition can remain as an emulsion for at least one year or at least two years. The ability of the composition to remain as an emulsion can be tested according to an accelerated stability test where the composition is held at 40° C. for 120 days. It is expected that this accelerated stability test for 120 days roughly corresponds to a period of about two years at room temperature. In general, it is expected that the composition can remain as an emulsion after sitting for two years at room temperature.

Exemplary surfactants that can be used as the surfactant component include nonionic surfactants that help stabilize the emulsion and provide a generally even distribution of the cationic pharmaceutically active ingredient containing component. Exemplary nonionic surfactants that can be used include glycerol stearate such as glycerol monostearate, polysorbate such as that available under the name Tween 80 and Polysorbate 60, polyoxyethylene stearate. In addition, mixtures of nonionic surfactants can be included including mixtures of polysorbate and glycerol stearate. An additional nonionic surfactant that can be used includes an ethoxy surfactant, a propoxy surfactant, or an ethoxy/propoxy surfactant. An exemplary ethoxy/propoxy surfactant includes a 10 carbon chain and 9 PO/EO surfactant available under the name Lutensol XP-90 from BASF. Additional nonionic surfactants include sorbitan monolaurate and sorbitan monostearate. Additional surfactants that can be used include those that are generally characterized as Pluronic surfactants such as poloxamers. Exemplary surfactants that can be used include Pluronic F-87NF and Pluronic L44NF from BASF.

It is believed that anionic surfactants may be useful as part of the surfactant component. In general, it is expected that anionic surfactants have a greater tendency to cause irritation to skin tissue.

The composition can include an amount of surfactant component sufficient to provide the composition with a desired emulsion stability and sufficiently low viscosity without foaming. The amount of the surfactant component in the composition, can be about 0.2 wt. % to about 7 wt. %, about 0.5 wt. % to about 6 wt. %, and about 1 wt. % to about 5 wt. %. It should be understood that the composition can be provided without any surfactant component, if desired.

The composition can contain a release agent to assist with the sustained release of the serine protease inhibitor over a prolonged period of time. A sustained release of the serine protease inhibitor refers to a release, over the time period, wherein the release provides desired properties. In general, it is desirable for the composition to provide a relatively consistent release of the serine protease inhibitor after application of the composition to skin tissue. A relatively consistent release can be characterized as a release rate at one hour that is within about 50% of the release rate at 30 minutes. In addition, a relatively consistent release rate can be characterized as a release rate at two hours that is within about 50% of the release rate at 30 minutes. Preferably, these release rates can be provided within about 25%, and more preferably can be provided within about 15%.

At least two advantages can be obtained by providing a sustained release rate or a relatively constant release rate over a prolonged period of time. For example, by providing a sustained release of the serine protease inhibitor over a prolonged period of time, it is possible to prolong the pharmaceutical efficacy of the composition after application to skin tissue. By prolonging the pharmaceutical efficacy of the composition, it is expected that enhanced performance can be achieved. Furthermore, by controlling the release of the serine protease inhibitor so that it is not released at one instant in time, it is possible to reduce or minimize skin irritation.

It is possible that the release agent of the ichthyosis treatment composition is not a surfactant. The ichthyosis treatment composition can include a non-surfactant release agent in an amount of about 0.2 wt. % to about 7 wt. %.

Emollient

The composition can include an emollient for improving the texture of the composition. An emollient is an oleaginous or oily substance which helps to smooth and soften the skin, and may also reduce its roughness, cracking or irritation. Exemplary suitable emollients include mineral oil, having a viscosity in the range of 50 to 500 centipoise (cps), lanolin oil, coconut oil, cocoa butter, olive oil, almond oil, macadamia nut oil, synthetic jojoba oils, natural sonora jojoba oils, safflower oil, corn oil, liquid lanolin, aloe vera, cottonseed oil, and peanut oil.

Other suitable emollients include squalane, castor oil, polybutene, odorless mineral spirits, sweet almond oil, avocado oil, clophyllum oil, ricin oil, vitamin E acetate, olive oil, linolenic alcohol, coconut oil, oleyl alcohol, the oil of cereal germs such as the oil of wheat germ, isopropyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glycerol, ricin oleates of alcohols and poly alcohols such as those of isopropyl adipate, hexyl laurate and octyl dodecanoate.

Other suitable emollients which are solids or semi-solids at room or ambient temperatures may be used in amounts sufficient to provide liquid topical compositions. Such solid or semi-solid cosmetic emollients include hydrogenated lanolin, hydroxylated lanolin, acetylated lanolin, petrolatum, isopropyl lanolate, butyl myristate, cetyl myristate, myristyl myrislate, myristyl lactate, cetyl alcohol, isostearyl alcohol and isocetyl lanolate. Exemplary emollients include stearic acid, stearyl alcohol, palmitic acid enters natural and synthetic esters such as coconut oil.

The composition can include the emollient in an amount sufficient to provide a silky feel. An exemplary range of the emollient in the composition can be at least about 0.5 wt. %. In addition, the composition can include an emollient in an amount of less than about 3 wt. %. It should be understood that the emollient is an optional component of the composition. The composition can be provided without an emollient, if desired.

Moisturizer

The composition can include a moisturizer to provide a desired moisturizing effect to skin tissue. The moisturizer can be provided as a humectant. In general, a humectant is a moistening agent that promotes retention of water due to its hydroscopic properties. Exemplary humectants include glycerine, polymeric glycols such as polyethylene glycol and polypropylene glycol, and sorbitols such as sorbitol solution, pyrrolidone carboxylic acid, urea, or mixtures thereof. The composition can be provided without a moisturizer.

When the composition includes a moisturizer, it can be included in an amount of at least about 0.5 wt. %. In addition, the composition can include a moisturizer in an amount of less than about 5 wt. %.

Skin Soothing Agent

An additional component that can be provided as part of the composition is a skin soothing agent or component which can also be referred to as a skin protectant. Exemplary skin soothing agents include allantoin, kaolin, cocoa butter, glycerine, shark liver oil, pertolatam, zinc oxide, zinc carbonate, zinc acetate, aluminum hydroxide, calamine, and mixtures thereof.

The skin soothing agent can be provided in the composition in an amount of about 0.2 wt. % to about 2 wt. %, and preferably about 0.5 wt. % to about 1 wt.5.

Preservatives

The composition can include preservatives for prevention of bacterial, fungal, and/or yeast contamination. Exemplary preservatives that can be used in the hand disinfecting composition include phenoxyethanol, benzoic acid, derivatives and salts of benzoic acid, parabens, oxazolidines, chlorinated aromatic compounds and phenols, hydantoins, cresols and derivatives, imiazolindinyl urea, iodopropanol butylcarbamate, sulfites, and bisulfites. The composition can include any of the preservatives commonly used or known to be suitable for topically applied compositions. Exemplary commercially available preservatives include liquid Germal Plus (diazolidinyl urea and iodopropynyl butylcarbamate) and Germaben 11 (diazolidinyl urea and methylparaben and propylparaben).

The composition can be formulated without a preservative. It is expected that the preservative will increase the shelf life of the composition by reducing or preventing the growth of bacteria, fungus, and/or yeast. When the composition includes a preservative, the preservative is preferably provided in an amount sufficient to provide a desired level of protection from growth of bacteria, fungus, and/or yeast.

In general, for most preservatives, it is expected that the amount of preservative can be provided at a level of about 0.1 wt. % to about 1.0 wt. %, and can be provided at a level of about 0.2 wt. % to about 0.5 wt. %, based on the weight of the composition.

Antioxidants

The composition can include antioxidants to help increase the shelf life of the composition and to provide desired properties when applied to skin tissue. Exemplary antioxidants that can be used include vitamins such as vitamin E, vitamin E acetate, vitamin C, and vitamin D, and derivatives thereof. Exemplary antioxidants include α-tocopherols which can be characterized as natural or synthetic Vitamin E. Additional exemplary antioxidants include propyl, octyl and dodecyl esters of gallic acid, butylated hydroxyanisole (BHA) (usually as a mixture of ortho and meta isomers), butylated hydroxytoluene (BHT), and nordihydroguaiaretic acid, and alkylated parabens such as methylparaben and propylparaben.

The composition can be formulated without an antioxidant. When the composition includes an antioxidant, the antioxidant can be provided in an amount that provides antioxidant properties in the composition. In general, it is expect that the antioxidant can be provided in an amount of about 0.2 wt. % to about 2 wt. %, and can be provided in an amount of about 0.7 wt. % to about 1.5 wt. %, based on the weight of the composition. In the case of vitamin E, it is expected that the vitamin E can be included in the composition in an amount of about 0.1 wt % to about 1 wt. %, and can be included in an amount of about 0.3 wt. % to about 0.8 wt. %.

Chelating Agents

Chelating agents are substances used to chelate or bind metallic ions with a certain heterocyclic ring structure so that the ion is held by chemical bonds from each of the participating rings. Suitable chelating agents include ethylene diaminetetraacetic acid (EDTA), EDTA trisodium, EDTA tetrasodium, calcium disodium edetate, EDTA trisodium, EDTA tetrasodium and EDTA dipotassium. One or more chelating agents can optionally be included in the emulsion in amounts ranging from about 0.001 to about 0.1 weight percent. It should be appreciated that the composition can be provided without a chelating agent.

Fragrances

Fragrances are aromatic compounds which can impart an aesthetically pleasing aroma to the composition. Typical fragrances include aromatic materials extracted from botanical sources (i.e. rose petals, gardenia blossoms, jasmine flowers, etc.) which can be used alone or in any combination to create essential oils. Alternatively, alcoholic extracts may be prepared for compounding fragrances. One or more fragrances can optionally be included in the composition in an amount ranging from about 0.001 to about 10 weight percent, preferably about 0.05 to about 5 percent. It should be appreciated that the composition can be provided without a fragrance.

Carriers, Diluents, and Excipients

The composition may also include non-toxic, pharmaceutically and dermatologically acceptable carriers, diluents and excipients, suitable for topical application, as are well known, see for example Merck Index, Merck & Co., Rahway, N.J., Bioreversible Carriers in Drug Design, Theory and Application, Roche (ed.) Pergamon Press, (1987), Gilman et al., (eds) (1990) Goodman and Gilman's: The Pharmacological Bases of Therapeutics, 8th Ed., Pergamon Press, Novel Drug Delivery Systems, 2nd Ed., Norris (ed.) Marcel Dekker Inc., (1989), and Remington's Pharmaceutical Sciences. For standard dosages of conventional pharmacological agents, see, e.g., Physicians Desk Reference (1997 Edition); and American Medical Association (1997) Drug Evaluations (Subscriptions).

The composition can be applied to skin tissue as a result of applying the composition as a cream or lotion to the skin tissue, and rubbing onto the skin tissue. The action of rubbing may include gentle rubbing or vigorous rubbing. Although the composition is sometimes characterized as having a binding or almost adhesive property with respect to skin tissue, the composition is not the type of composition that one would consider to be an adhesive that holds two substrates together such as, for example, a hot melt adhesive.

Because the composition can bind serine protease inhibitor therein, the release of the serine protease inhibitor can take place over time.

The above specification, examples and data provide a complete description of the manufacture and use of the composition of the invention. Since many embodiments of the invention can be made without departing from the spirit and scope of the invention, the invention resides in the claims hereinafter appended.

### Example 1

An exemplary composition is provided as a spray dermatitis treatment or diaper rash treatment composition, and is presented in Table 1.

TABLE 1
Diaper Rash Spray Composition

| Components | | wt. % |
|---|---|---|
| Water | | 86.10 |
| Carbopol Ultrez-10 | Thickener | 0.30 |
| Polysorbate 60 | Release agent | 1.00 |
| DPHP | Inhibitor | 1.00 |
| Dow Corning 9045 | Barrier Component | 1.00 |
| Invisicare^{tm} C-5 | Skin Bonding | 6.00 |
| Composition | Polymer | |
| Allantoin | Soothing Agent | 0.50 |
| Stearic Acid | Emollient | 1.00 |
| Coconut Oil | Emollient | 2.00 |
| Triethanolamine | pH adjusting agent | 0.60 |
| Phenoxyethanol | Preservative | 0.50 |

Because the composition exhibits barrier properties, it is not necessary to release the active ingredient from the composition. Dual action of this product stems from a) prevention of contact due to the barrier properties of the composition, and b) inhibition of serine proteases coming in contact with the barrier. The product also includes allantoin as a skin soothing agent as well as the emollients stearic acid and coconut oil to promote general skin health.

As stated above, the body generally produces proteases and inhibitors in balance. Skin aging is an example of the result of imbalance between a protease and an inhibitor. Such an imbalance can also produce disease.

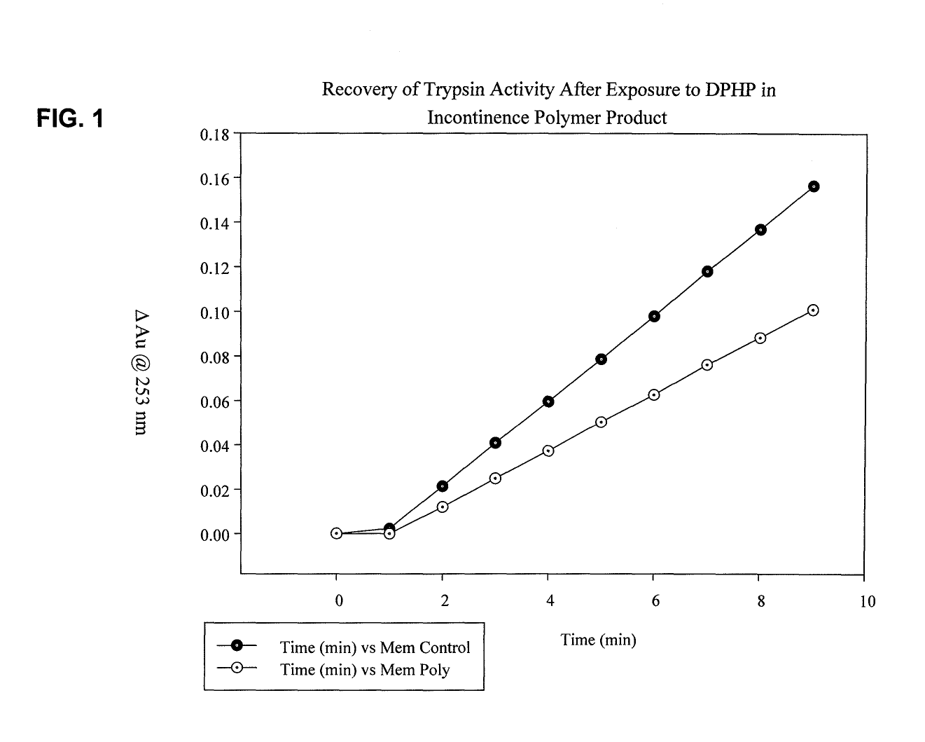

The graph in FIG. 1 demonstrates a 29.14% reduction in trypsin activity after exposure to the diaper rash spray product of Example 1.

Serine proteases (those proteolytic enzymes having serine in the active site) are ubiquitous in nature. Serine proteases are secreted by bacteria as exogenous digestive enzymes, also as digestive enzymes by plants, fungi and mammals.

Trypsin is a typical serine protease. It is produced in the pancreas of mammals, and released into the small intestine to digest proteins so they may be absorbed by the body. Trypsin, at least in part, survives the digestive process and is present in animal feces. Trypsin has been well characterized, and is available in a pure form, usually isolated from bovine pancreas. Since Trypsin and bacteria are both present in human feces, their ability to digest the proteins in skin represent a source of irritation to the skin of babies and the incontinent. Continued presence of proteolytic activity on the skin results in a dermatitis commonly referred to as diaper rash.

Clearly the presence of a protease inhibitor in a skin protectant barrier product would decrease the susceptibility of the skin to diaper rash in infants and the incontinent. Several such serine protease inhibitors are known. Many of these serine protease inhibitors are naturally occurring in the body, and are necessary for normal bodily functions, such as blood clotting etc.

The most common protease inhibitor for trypsin is soybean trypsin inhibitor, that, as the name implies, is present in raw soybeans. Unfortunately, soybean trypsin inhibitor is a relatively high molecular weight protein (170 to 200 amino acid residues, 18,700 to 22,000), that is not stable for extended periods of time at room temperature or above. It is easily destroyed by the roasting of soybeans. Chemical inhibitors of serine proteases are generally too toxic or too reactive to be placed on the skin.

Dipalmitoylhydroxyproline (DPHP) is currently marketed in anti-aging products because it inhibits the elastase and certain collagenases that breakdown the supporting elastin and collagen in skin as it ages. The breakdown of elastin and collagen in the skin is what results in fine lines and wrinkles in aging skin. These elastases and collagenases are serine proteases.

DPHP can be included in a skin soothing and protecting barrier product. The data in FIG. 1 demonstrates that DPHP can significantly reduce proteolytic activity of exogenously administered proteases. This is a significant step forward in treating and preventing diaper rash in the incontinent.

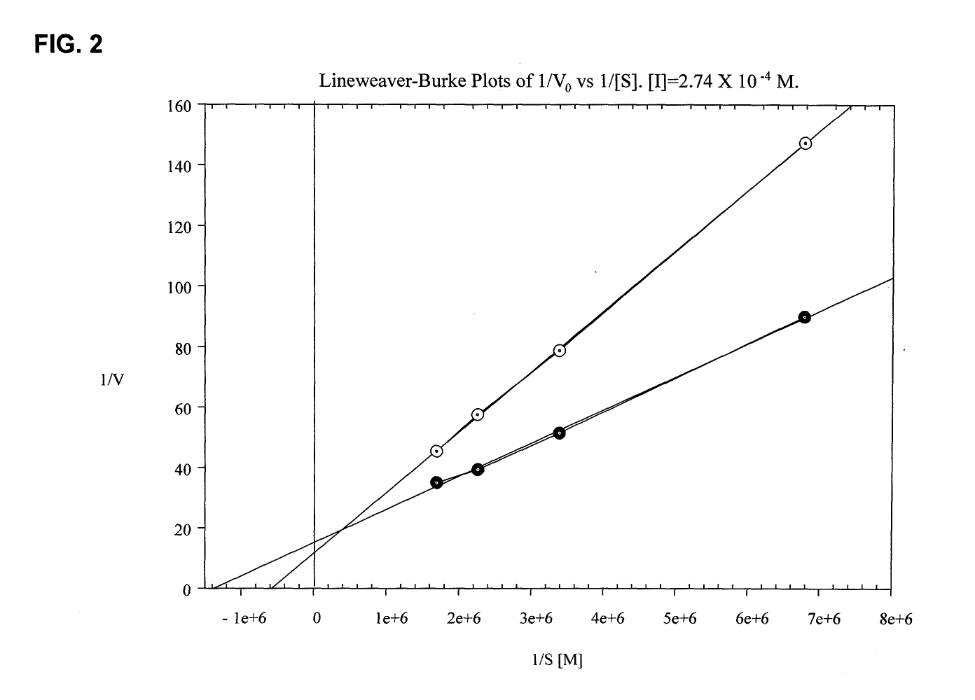

The graph shown in FIG. 2 is a Lineweaver-Burk plot of uninhibited Trypsin (the lower line after zero) using N-Benzoyl-L-Arginine ethylester as a substrate and the inhibition of trypsin by DPHP (the upper line after zero). The graph demonstrates that DPHP is a competitive inhibitor of trypsin, and hence the inhibition is reversible by dilution or addition of more substrate.

This reversible type of enzyme inhibition is important to understanding the efficacy data shown on page 1 of this document. When applied to the membrane containing the DPHP, the trypsin may have been completely inhibited, but the process of eluting the trypsin from the membrane, and subsequently measuring its activity, reduced the levels of available inhibitor, and the trypsin activity appears higher than it may actually have been.

The two products described in this patent differ in nature because of the location of the enzymes to be inhibited. In the case of the incontinence product, the proteolytic enzymes are external to the skin, and it is only necessary for the inhibitor to be bound to a protective film on the skin. Any proteolytic enzyme coming in contact with the film will be inhibited.

### Example 2

A skin disorder treatment composition is shown in Table 2. The treatment composition includes an inhibitor that is to be released from the product after application so that it can penetrate the skin and inhibit the enzyme. For these reasons, the formula for the composition, shown below, differs from the incontinence formulation.

TABLE 2
Treatment Cream

| Components | | wt. % |
|---|---|---|
| Water | | 84.90 |
| Carbopol Ultrez-10 | Thickener | 0.45 |
| Polysorbate 60 | Release agent | 1.50 |
| DPHP | Inhibitor | 1.00 |
| EDTA | Chelating agent | 0.05 |
| Pluronic F-87NF | Release agent | 1.20 |
| Invisicare^{tm} M-1 | Skin bonding | 5.00 |
| Composition | Polymer component | |
| Allantoin | Skin soothing agent | 0.50 |
| Stearic Acid | Emollient | 1.00 |
| Coconut Oil | Emollient | 2.00 |
| Triethanolamine | pH adjusting agent | 1.90 |
| Phenoxyethanol | Preservative | 0.50 |

It is desirable for the treatment composition to provide a release of about 40% DPHP over 2 hours. In general, the performance of a treatment composition can be characterized as a composition that provides a release of at least 40 wt. % serine protease inhibitor over 2 hours. Preferably, the release is relatively sustained. That is, the release preferably does not all occur within 10 minutes, but is provided relatively evenly over at least a couple hours. The composition of Table 2 has been tested and a release of at least 40% DPHP over 2 hours has been observed, and the release is relatively sustained over the 2 hours.

The composition reported in Table 2 can be used to treat ichthyosis, dermatitis, eczema, rosacea, psoriasis, inflammation, xerosis, and urticaria, or skin tissue suffering from symptoms of ichthyosis, dermatitis, eczema, rosacea, psoriasis, inflammation, xerosis, or urticaria.

Preferred embodiments are described herein, including the best mode known to the inventor for carrying out the invention. Variations of those preferred embodiments may become apparent to those of ordinary skill in the art upon reading the forgoing description. The inventor expects skilled artisans to employ such variations as appropriate, and the invention to be practiced otherwise than as specifically described herein. Accordingly, the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A dermatitis treatment composition comprising:
(a) about 0.0001 wt. % to about 5 wt. % of a serine protease inhibitor;
(b) about 2 wt. % to about 10 wt. % of a hydrophobic polymer/hydrophilic polymer complex comprising a poly(vinylpyrrolidone-alkylene) polymer wherein the alkylene group contains at least 10 carbon atoms and a cellulose containing polymer;
(c) about 0.5 wt. % to about 20 wt. % of a barrier component; and
(d) at least about 50 wt. % water.

2. A dermatitis treatment composition according to claim 1, wherein the poly(vinlypyrrolidone-alkylene) polymer comprises a polymer having an alkylene group containing about 10 carbon atoms to about 30 carbon atoms.

3. A dermatitis treatment composition according to claim 1, wherein the composition comprises about 0.5 wt. % to about 6 wt. % of a surfactant.

4. A dermatitis treatment composition according to claim 1, wherein the composition comprises about 0.1 wt. % to about 2 wt. % of a thickener.

5. A dermatitis treatment composition according to claim 1, wherein the composition comprises about 0.5 wt. % to about 5 wt. % of a moisturizer.

6. A dermatitis treatment composition according to claim 1, wherein the composition has a pH of about 3.5 to about 7.

7. A dermatitis treatment composition according to claim 1, wherein the serine protease inhibitor comprises dipalmitoylhydroxyproline.

8. A method for using a dermatitis treatment composition according to claim 1, the method comprising applying the dermatitis treatment composition to skin tissue.

9. An ichthyosis treatment composition comprising:
(a) about 0.0001 wt. % to about 5 wt. % of a serine protease inhibitor;
(b) about 2 wt. % to about 10 wt. % of a hydrophobic polymer/hydrophilic polymer complex comprising a poly(vinylpyrrolidone-alkylene) polymer wherein the alkylene group contains at least 10 carbon atoms and a poly (maleic acid/methylvinylether) copolymer;
(c) about 0.2 wt. % to about 7 wt. % of a release agent; and
(d) at least about 50 wt. % water.

10. An ichthyosiss treatment composition according to claim 9, wherein the poly(vinlypyrrolidone-alkylene) polymer comprises a polymer having an alkylene group containing about 10 carbon atoms to about 30 carbon atoms.

11. An ichthyosis treatment composition according to claim 9, wherein the release agent comprises a surfactant.

12. An ichthyosis treatment composition according to claim 9, wherein the composition comprises about 0.5 wt. % to about 6 wt. % of a surfactant.

13. An ichthyosis treatment composition according to claim 9, wherein the composition comprises about 0.1 wt. % to about 2 wt. % of a thickener.

14. An ichthyosis treatment composition according to claim 9, wherein the composition comprises about 0.5 wt. % to about 5 wt. % of a moisturizer.

15. An ichthyosis treatment composition according to claim 9, wherein the composition has a pH of about 3.5 to about 7.

16. An ichthyosis treatment composition according to claim 9, wherein the serine protease inhibitor comprises dipalmitoylhydroxyproline.

17. A method for treating ichthyosis utilizing the ichthyosis treatment composition according to claim 9, wherein the method comprising a step of applying ichthyosis treatment composition to skin tissue.

18. A composition for treating a skin disorder or symptoms of a skin disorder comprising:
(a) about 0.0001 wt. % to about 5 wt. % of a serine protease inhibitor;
(b) about 2 wt. % to about 10 wt. % of a hydrophobic polymer/hydrophilic polymer complex comprising a poly(vinylpyrrolidone-alkylene) polymer wherein the alkylene group contains at least 10 carbon atoms and a poly (maleic acid/methylvinylether) copolymer;
(c) about 0.2 wt. % to about 7 wt. % of a release agent; and
(d) at least about 50 wt. % water.

19. A composition according to claim 17, wherein the skin disorder or the symptom of a skin disorder comprises at least one of ichthyosis, dermatitis, eczema, rosacea, psoriasis, xerosis or urticaria.

20. A method for treating a skin disorder utilizing a composition according to claim 19, wherein the method comprises the step of applying the composition to skin tissue.
